(19) United States
(12) Patent Application Publication
(10) Pub. No.: US 2016/0143845 A1
(43) Pub. Date: May 26, 2016

(54) QUICK DISSOLVE NUTRITIONAL POWDER

(71) Applicant: Rich Vitamins LLC, New York, NY (US)

(72) Inventors: Hallie N. Rich, New York, NY (US); Michelle G. Cannon, Ithaca, NY (US)

(21) Appl. No.: 15/001,302

(22) Filed: Jan. 20, 2016

Related U.S. Application Data
(63) Continuation-in-part of application No. 13/583,698, filed on Oct. 24, 2012, now abandoned.
(60) Provisional application No. 61/312,784, filed on Mar. 11, 2010.

(51) Int. Cl. A61K 9/00; A61K 33/30; A61K 31/07; A61K 31/375; A61K 31/59; A61K 31/355; A61K 31/455; A61K 33/26; A61K 33/32; A61K 33/24; A61K 33/22; A61K 33/06; A61K 33/10; A61K 45/06; A61K 33/08; A61K 33/34

(52) U.S. Cl. CPC A61K 9/0056; A23L 1/296; A23L 1/302; A23L 1/303; A23L 1/304; A23L 1/3045; A23L 1/236; A23L 1/2363; A23L 1/2362; A61K 33/34; A61K 33/30; A61K 31/07; A61K 31/375; A61K 31/59; A61K 31/355; A61K 31/455; A61K 33/26; A61K 33/32; A61K 33/24; A61K 33/22; A61K 33/06; A61K 33/10; A61K 45/06; A61K 33/08; A23V 2002/00

(57) ABSTRACT

The present disclosure provides a rapidly disintegrating orally administrable powder. The powder may include an active ingredient such as a dietary supplement. Methods of using the same are also provided herein.

### CROSS-REFERENCE TO RELATED APPLICATIONS

This application is a continuation-in-part application of U.S. patent application Ser. No. 13/583,698, filed on Sep. 10, 2012, which is a U.S. National Stage Application of International Application No. PCT/US2011/027827 filed on Mar. 10, 2011 which, in turn, claims the benefit of and priority to U.S. Provisional Patent Application Ser. No. 61/312,784 filed on Mar. 11, 2010, the entire disclosures of each of which are hereby incorporated by reference herein.

### BACKGROUND

The present disclosure relates to a powered dosage form of dietary supplement that rapidly dissolves on the tongue.

Dietary supplementation with vitamins, minerals, herbs, and other dietary ingredients is within the purview of those skilled in the art. These supplements may be used to assist a person in meeting certain dietary needs. However, many dietary supplements have a flavor and/or mouthfeel, which make them unpalatable. Additionally, some dietary supplements leave an unpleasant aftertaste in the mouth.

Numerous methods of administering these supplements have been developed, such as swallowable and chewable tablets, hard and soft shell capsules, effervescent wafers, liquids, gummy chews, sublingual pellets, and the like. However, many individuals find it difficult to chew gummy chews or chewable tablets, or difficult to swallow tablets, capsules, or large quantities of liquids, while others object to the high refined sugar content or low nutritional content of such dosage forms.

Improved methods of providing palatable dietary supplements, which possess good mouthfeel and do not leave an unpleasant aftertaste, remain desirable. Additionally, alternative methods of administration that do not require swallowing a tablet, pill, capsule, and the like, or chewing a chewable dosage form, remain desirable.

### SUMMARY

The present disclosure provides a rapidly dissolving orally administrable palatable powder. The powder may include an edible polyol, an active ingredient such as calcium, at least one natural flavor, a sweetening ingredient, a disintegrant, and an organic acid.

The present disclosure also provides a rapidly dissolving orally administrable powder including an edible polyol in an amount of from about 20% to about 80% by weight of the powder, an active ingredient in an amount of from about 10% to about 50% by weight of the powder, a flow agent in an amount of from about 0% to about 2% by weight of the powder, at least one natural flavor in an amount of from about 3% to about 15% by weight of the powder, a sweetening ingredient in an amount of from about 0.5% to about 25% by weight of the powder, an organic acid in an amount of from about 0.5% to about 5% by weight of the powder, and a disintegrant in an amount of from about 4% to about 20% by weight of the powder.

Methods of producing and administering dietary supplements are also provided herein. In embodiments, a method of the present disclosure includes dry blending a mixture including a polyol, an active ingredient, at least one natural flavor, a sweetening ingredient, an organic acid, and a disintegrant to form a rapidly dissolving orally administrable palatable powder. The palatable powder may be orally administered. The powder rapidly dissolves in the mouth, in embodiments, on the tongue.

In other embodiments, compositions of the present disclosure may include a powder including at least one bulking agent; at least one sweetening ingredient; and at least one active ingredient selected from the group consisting of Vitamin A, Vitamin C, Vitamin D, Vitamin E, Thiamin, Riboflavin, Niacin, Vitamin B6, Folate, Vitamin B12, Pantothenic Acid, Biotin, Chromium, Copper, Iodine, Iron, Manganese, Molybdenum, Selenium, Zinc, Boron, omega-3 fatty acids, coenzyme Q10, calcium, probiotics, and combinations thereof, wherein the powder dissolves in the mouth upon contact with saliva.

Still other compositions of the present disclosure may include, in embodiments, a powder including at least one bulking agent in an amount of from about 40% to about 80% by weight of the powder; at least one active ingredient in an amount of from about 10% to about 50% by weight of the powder; calcium in an amount from about 12% to about 18% by weight of the powder; at least one flow agent in an amount of from about 0% to about 2% by weight of the powder; at least one sweetening ingredient in an amount of from about 0.5% to about 25% by weight of the powder; at least one organic acid in an amount of from about 0.5% to about 5% by weight of the powder; and at least one soluble fiber in an amount of from about 1% to about 25% by weight of the powder, wherein the powder dissolves in the mouth upon contact with saliva.

In other embodiments, methods of the present disclosure may include preparing a dietary supplement by dry blending a mixture comprising at least one bulking agent, at least one sweetening ingredient, and an active ingredient selected from the group consisting of Vitamin A, Vitamin C, Vitamin D, Vitamin E, Thiamin, Riboflavin, Niacin, Vitamin B6, Folate, Vitamin B12, Pantothenic Acid, Biotin, Chromium, Copper, Iodine, Iron, Manganese, Molybdenum, Selenium, Zinc, Boron, omega-3 fatty acids, coenzyme Q10, calcium, probiotics, and combinations thereof, to form a rapidly dissolving orally administrable palatable powder; introducing the orally administrable powder into a mouth of a consumer; and allowing the orally administrable powder to dissolve in the mouth.

### DETAILED DESCRIPTION

The present disclosure provides a dosage form and method of dietary supplementation that is palatable, providing a pleasant flavor and mouthfeel, and does not produce an unpleasant aftertaste. The dosage form of the present disclosure is an oral unit that rapidly dissolves in the mouth and/or on the tongue. The dosage form of the present disclosure may be in powder form or granules. Granular forms may be obtained where a process is used to make particle sizes larger, such as by agglomeration. The dosage and method of administration do not require swallowing of a solid or liquid dosage form, or chewing of tablets or chewable dosage forms.

The present dosage form is a rapidly dissolving orally administrable palatable powder. As used herein, the term “rapidly dissolving orally administrable palatable powder” includes non-liquid preparations that disintegrate when placed in the mouth upon contact with saliva in the oral cavity, in embodiments on the tongue. The powders may disintegrate within a period of time from about 2 seconds to about 30 seconds, in embodiments from about 5 seconds to about 20 seconds. The rate of disintegration, sometimes referred to herein as the dissolution rate, may depend upon the level of saliva in the oral cavity, as well as the amount of powder that is poured on the tongue. As used herein, the term “powder” may include a powder, crystalline materials, microgranulated materials, granulated materials, agglomerates, adsorbates, microencapsulated materials, encapsulated materials, combinations thereof, and the like.

The rapidly dissolving orally administrable palatable powder may include, for example, at least one bulking agent; active ingredients; natural flavors; sweetening ingredients; disintegrants; organic acids; flow agents; combinations thereof, and the like.

In embodiments, the rapidly dissolving orally administrable palatable powder includes at least one bulking agent. Such bulking agents include, for example polyols, sucrose, maltodextrin, combinations thereof, and the like. As used herein, a “bulking agent” is any inert additive that increases the volume of the orally administrable palatable powder of the present disclosure, without affecting its taste. Bulking agents may improve the mouth feel of the orally administrable palatable powder of the present disclosure, and are of acceptable physical characteristics to achieve blend uniformity and uniformity in appearance.

In embodiments, any polyol that is edible may be used as a bulking agent in accordance with the present disclosure. In embodiments, the polyol may be, for example, sorbitol, maltitol, erythritol, isomalt, lactitol, polyglycitol, polydextrose, mannitol, xylitol, combinations thereof, and the like. In embodiments, the polyol includes mannitol in combination with xylitol.

In embodiments, the bulking agent may be present in the orally administrable powder in an amount of from about 20% to about 85% by weight of the powder, in embodiments from about 40% to about 80% by weight of the powder, in other embodiments from about 30% to about 70% by weight of the powder.

The bulking agent may be of low hygroscopicity. In embodiments, the bulking agent is marginally hygroscopic, taking up water above a relative humidity of about 79%. In other embodiments, the edible polyol is non-hygroscopic, only taking up water when exposed to a relative humidity of greater than about 97%. As noted above, in embodiments combinations of bulking agents may be used, including those that are marginally hygroscopic with those that are non-hygroscopic.

Where the bulking agent is a polyol, the polyol may be a flowable granulated or powdered polyol. For example, in embodiments, mannitol may be used. Mannitol is non-hygroscopic, taking up water only above a relative humidity (RH) of about 97% RH. The mannitol may be a granulated grade of mannitol, with from about 90% to about 95% of the particles having a particle size of from about 210 to about 450 microns, with an approximate mean particle size of about 320 microns. In other embodiments, the mannitol may be a powdered grade, with from about 80% to about 90%, in embodiments about 85%, of the particles having a particle size from about 40 to about 500 microns, with a mean particle size of about 150 microns. At about 37° C., from about 22% to about 23% of the mannitol goes into solution.

As noted above, xylitol may be used as a polyol. In embodiments, xylitol may be used in combination with mannitol. Xylitol is a non-cariogenic sweetening agent, odorless, and is marginally hygroscopic. It has the same sweetening power as sucrose and provides a distinct cooling effect in the mouth, which is effective in enhancing flavor and masking any unpleasant or bitter flavors associated with some actives. Xylitol helps increase the flow of saliva, which enhances the rapid dissolution of the supplement. Where xylitol is used, the xylitol may be in a crystalline powder form. Most of the xylitol particles will have a particle size of from about 120 microns to about 370 microns, with an approximate mean particle size of about 250 microns. Xylitol is more soluble than mannitol, so about 62.5% of the xylitol dissolves in water at about 20° C., and about 65% or more goes into solution at about 37° C.

Any dietary ingredient may be used as an active ingredient of the rapidly dissolving orally administrable powder. The active ingredient may be, for example, vitamins, minerals, herbs, and other dietary ingredients, as well as combinations thereof. Vitamins that may be used in accordance with the present disclosure include, for example: vitamin A from sources such as vitamin A palmitate, vitamin A acetate, beta carotene, combinations thereof, and the like; vitamin C from sources such as sodium ascorbate, ascorbic acid, calcium ascorbate, combinations thereof, and the like; vitamin D from sources such as ergocalciferol (D2), cholecalciferol (D3), combinations thereof, and the like; vitamin E from sources such as d-α-tocopheryl acetate, d-α-tocopherol, d-α-tocopheryl succinate, dl-α-tocopherol, dl-α-tocopheryl succinate, dl-α-tocopheryl acetate, combinations thereof, and the like; thiamin (vitamin B1), thiamin mononitrate, combinations thereof, and the like; riboflavin (vitamin B2), riboflavin 5′ monophosphate, combinations thereof, and the like; niacin (vitamin B3) from sources such as niacinamide, nicotinic acid, inositol hexanicotinate, combinations thereof, and the like; vitamin B6 from sources such as pyridoxine hydrochloride, pyridoxal-5′-phosphate, combinations thereof, and the like; folate from sources such as folic acid, folinic acid, combinations thereof, and the like; vitamin B12 from sources such as cyanocobalamin, methylcobalamin, combinations thereof, and the like; pantothenic acid (vitamin B5) from sources such as calcium d-pantothenate, pantetheine, pantothenol, sodium pantothenate, combinations thereof, and the like; biotin from sources such as biotin trituration and brewer's yeast, combinations thereof, and the like.

Minerals that may be used in accordance with present disclosure include, for example: calcium from sources such as calcium carbonate, calcium citrate, calcium aspartate, calcium gluconate, calcium lactate, calcium lactate gluconate, calcium citrate malate, dicalcium malate, combinations thereof, and the like; chromium from sources such as chromium polynicotinate, chromium chloride, chromium-enriched yeast, chromium picolinate, chromium amino acid chelate, combinations thereof, and the like; copper from sources such as copper amino acid chelate, copper gluconate, copper sulfate, copper oxide, copper carbonate, combinations thereof, and the like; iodine from sources such as potassium iodide, ammonium iodide, calcium iodide, kelp, combinations thereof, and the like; iron from sources such as iron amino acid chelate, ferrous fumarate, ferrous succinate, ferrous sulfate, ferrous gluconate, ferritin, ferric orthophosphate, combinations thereof, and the like; magnesium from sources such as magnesium oxide, magnesium carbonate, magnesium amino acid chelate; magnesium citrate, magnesium dolomite, magnesium aspartate, magnesium fumarate, magnesium hydroxide, dimagnesium malate, magnesium aspartate, combinations thereof, and the like; manganese from sources such as manganese amino acid chelate, manganese sulfate, manganese gluconate, manganese chloride, combinations thereof, and the like; molybdenum from sources such as sodium molybdate, molybdenum amino acid chelate, combinations thereof, and the like; selenium from sources such as selenium yeast, selenomethionine, sodium selenite, combinations thereof, and the like; zinc from sources such as zinc oxide, zinc gluconate, zinc sulfate, zinc picolinate, zinc amino acid chelate, zinc chloride, combinations thereof, and the like; boron from sources such as boron amino acid complex, sodium borate, combinations thereof, and the like.

Any other active ingredient within the purview of those skilled in the art may be included with the powder dosage form of the present disclosure. For example, in embodiments, coenzyme Q10, omega-3 fatty acids, and/or probiotics may be included in a powder dosage form of the present disclosure.

Active ingredients, for example those described above, may be provided and/or obtained in any form within the purview of those skilled in the art. The active ingredients described above may also be obtained from any other source within the purview of those skilled in the art, including commercially available sources, and/or synthesized by methods within the purview of those skilled in the art.

In embodiments, the rapidly dissolving orally administrable powder may include a combination of active ingredients. The amount of each active ingredient included in the rapidly dissolving orally administrable powder may vary depending on the number of active ingredients and the type of other active ingredients included. In embodiments, the amount of active ingredient(s) in the rapidly dissolving orally administrable powder may be from about 10% to about 50% by weight of the powder, in embodiments from about 20% to about 45% by weight of the powder, in embodiments about 43% by weight of the powder.

In embodiments, the active ingredient may include from about 1 to about 30 active ingredients, in embodiments from about 3 to about 23 active ingredients. The amount of active ingredients included in a powdered dosage form of the present disclosure may be determined utilizing means within the purview of those skilled in the art including, for example, the Recommended (or Reference) Daily Intake (RDI). The RDI provides a reference amount of vitamins and minerals for consumption by most healthy individuals as a function of total daily caloric intake. In embodiments, the active ingredient may include from about 10% to about 1500% of the RDI of a certain vitamin or mineral, in embodiments from about 50% to about 750% of the RDI of a certain vitamin or mineral. The National Academy of Sciences (NAS) Tolerable Upper Intake Level should be considered when formulating with certain nutrients. For example, for some nutrients like niacin, which has an upper limit of 20-35 mg/day, the dosage form should not include more than about 175% of the RDI (the RDI for niacin is 20 mg).

The powder of the present disclosure may contain excipients, sometimes referred to herein as non-active ingredients, which may contribute to the powder's dissolution rate, pleasing mouthfeel, and pleasant taste. Suitable excipients include sweetening ingredients, flow agents, disintegrants, encapsulants, colorants, artificial flavors, combinations thereof, and the like.

In embodiments, the rapidly dissolving orally administrable powder may include a sweetening ingredient. The sweetening ingredient used may be a nutritive sweetening ingredient, a non-nutritive sweetening ingredient, or combinations of these. Nutritive sweetening ingredients that may be used in accordance with the present disclosure include, for example, thaumatin, sucrose, fructose, maltose, dextrose, combinations thereof, and the like. Non-nutritive sweetening ingredients that may be used in accordance with the present disclosure include, for example, stevia, sucralose, aspartame, saccharin, acesulfame K, combinations thereof, and the like. In embodiments, the amount of sweetening ingredient in the rapidly dissolving orally administrable powder may be from about 0.5% to about 25% by weight of the powder, in embodiments from about 1% to about 20% by weight of the powder.

Calcium carbonate may be added to a powder of the present disclosure, either as part of the active ingredients described above, or as an additional additive to impart improved mouthfeel, minimal flavor, and free flowing characteristics. Suitable sources of calcium carbonate include, for example, those commercially available as DESTAB calcium carbonate, from Particle Dynamics, which provides excellent mouthfeel, minimal flavor, desirable free flowing characteristics, and high calcium load. This ingredient has excellent dissolution in the mouth, and further improves the physical characteristics of the entire finished product. The calcium carbonate may include a binder, such as maltodextrin, which surrounds the calcium into a spherical particle with excellent flow and reduced chalkiness. Other sources of calcium carbonate, including those commercially available from Particle Dynamics that are spray dried with gum acacia, may be utilized.

The rapidly dissolving orally administrable powder possessing calcium may thus have an improved mouthfeel. The amount of the calcium source present in the powder may directly contribute to the mouthfeel of the powder. In embodiments, an amount of calcium source of about 15% by weight of the powder may provide excellent mouthfeel. Amounts of calcium source of less than about 8% by weight of the powder may provide reduced mouthfeel and slower oral dissolution. Amounts greater than about 23% by weight of the powder may require an increase in dosage size and result in reduced sweetness and flavor perception. Thus, suitable amounts of calcium source may be from about 8% to about 23% by weight of the powder, in embodiments from about 11 to about 18% by weight of the powder, in embodiments about 15% by weight of the powder. In embodiments, the amount of calcium may be from about 11% to about 23% by weight of the powder.

In embodiments, the rapidly dissolving orally administrable powder may include a dietary fiber. Dietary fibers include the edible parts of plants or similar carbohydrates, resistant to digestion and absorption in the human small intestine with complete or partial fermentation in the large intestine. Dietary fibers include both soluble and insoluble fibers. As used herein, a “soluble fiber,” when dispersed with water, forms a gel-like substance and swells (to varying degrees). The scientific names for soluble fibers may include pectins, gums, mucilages, resistant maltodextrin, inulin, oligosaccharides, polydextrose, and some hemicelluloses. Good sources of soluble fibers may include cereals (i.e., oats and oatmeal beta glucans) seeds (i.e., flaxseeds, chia seeds), psyllium, legumes (i.e., peas, beans, lentils), barley, chicory root and alternative sources of inulin, fruits and vegetables (i.e., asparagus, brussel sprouts, oranges, apples and carrots).

In embodiments, suitable sources of soluble fiber for use in forming the rapidly dissolving orally administrable powder of the present disclosure may include resistant starches, maltodextrin, oligosaccharides, inulin, gums, polydextrose, combinations thereof, and the like.

Suitable sources of soluble fiber which may be utilized include, for example, those soluble fibers commercially available as FIBERSOL®-2 soluble dietary fiber (from Matsutani America, Inc.). FIBERSOL®-2 soluble dietary fiber is maltodextrin, and is commercially available as both agglomerated and non-agglomerated forms.

Suitable sources of dietary fiber, in embodiments soluble fiber, are low in calories and may include from about 50% to about 95% soluble fiber, in embodiments about 90% soluble fiber.

Where utilized, dietary fiber(s) may be included in the rapidly dissolving orally administrable powder in amounts from about 1% to about 25% by weight of the rapidly dissolving orally administrable powder, in embodiments from about 3% to about 22% by weight of the rapidly dissolving orally administrable powder.

Soluble fibers may be added to impart various desirable properties to the rapidly dissolving orally administrable powder including, but not limited to, quicker oral dissolution of the powder and improved mouthfeel, without imparting any additional flavor or taste to the rapidly dissolving orally administrable powder. Other desirable properties that soluble fibers may impart to the rapidly dissolving orally administrable powder include: low hygroscopicity, which reduces caking; fiber delivery, which is a health benefit of itself; low dusting; very little effect on viscosity; flavor masking, for example, reducing the perception of metallic flavors which may be present where vitamins and/or minerals are included in the powder of the present disclosure; low bulk density (especially agglomerated versions); heat stability; and acid stability.

Magnesium oxide may be added to a powder of the present disclosure as part of the active ingredients described above, to impart improved minimal flavor and free flowing characteristics. Suitable sources of magnesium oxide include, for example, those commercially available as DESCOTE magnesium oxide, from Particle Dynamics, which has minimal flavor, a relatively free flowing nature, and high magnesium load. The magnesium oxide may be microencapsulated, in embodiments in a matrix including mono-and diglycerides, which may provide superior taste and minimal reactivity.

Magnesium, for example magnesium oxide, may be present in amounts of from about 0% to about 10% by weight of the powder, in embodiments from about 3% to about 8% by weight of the powder.

Viscosity-enhancing gums or polymers, such as, for example xanthan gum, guar gum, gum arabic, and the like, are not suitable in accordance with the present disclosure.

The rapidly dissolving orally administrable palatable powder of the present disclosure may include a disintegrant. Disintegrants that may be used in accordance with the present disclosure include, for example, polyvinyl pyrrolidone, crosscarmellose sodium, sodium starch glycolate, microcrystalline cellulose, starch, and combinations of these. In embodiments, the disintegrant may be polyvinyl pyrrolidone. Polyvinyl pyrrolidone has a smooth mouthfeel and rapid disintegration. In embodiments, the disintegrant particles are porous which allows for better wicking of liquid (including saliva), and of small size, which may provide excellent mouthfeel. In embodiments, the disintegrants have an average particle size of from about 20 to about 120 microns, in embodiments from about 30 to about 110 microns, in other embodiments about 30 microns.

In embodiments, the disintegrant is present in the rapidly dissolving orally administrable palatable powder form in an amount from about 4% to about 20% by weight of the powder, in embodiments from about 6% to about 18% by weight of the powder.

The rapidly dissolving orally administrable palatable powder of the present disclosure may also include an organic acid. Organic acids that may be used in accordance with the present disclosure include, for example, citric acid, fumaric acid, adipic acid, tartaric acid, acetic acid, lactic acid, phosphoric acid, sodium acid sulfate, malic acid, combinations thereof, and the like. The organic acid may be used to enhance any natural flavor added to the rapidly dissolving orally administrable powder. In embodiments, the amount of organic acid present in the rapidly dissolving orally administrable powder may be from about 0.5% to about 5% by weight of the powder, in embodiments from about 0.75% to about 4% by weight of the powder.

In embodiments, the rapidly dissolving orally administrable palatable powder may further include a flow agent. A flow agent may be added, for example, to enhance flow of the powder during manufacturing. Flow agents that may be used in accordance with the present disclosure include, for example, sodium bicarbonate, sodium ferrocyanide, potassium ferrocyanide, calcium ferrocyanide, bone phosphate, sodium silicate, silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane, combinations thereof, and the like. The flow agent may be present in the rapidly dissolving orally administrable palatable powder in an amount from 0% to about 2% by weight of the powder, in embodiments from about 0.25% to about 1.5% by weight of the powder.

In embodiments, an encapsulant may be included with any of the above active ingredients of the powder, including vitamins and minerals provided as dietary ingredients in the powder of the present disclosure. Suitable encapsulants include, for example, mono-glycerides, di-glycerides, fatty acids such as stearic acid, combinations thereof, and the like. The encapsulant may be applied to the ingredient(s), for example, to enhance shelf life, mask flavor, act as a physical barrier to protect the active component and thus minimize its reactivity, and combinations of these.

For example, vitamins such as Vitamin B1, B2, and B6 may be encapsulated with mono-glycerides and/or diglycerides, in amounts from about 55% to about 80% by weight of the vitamin, in embodiments from about 60% to about 75% by weight of the vitamin. Similarly, niacin (vitamin B3) may be encapsulated with stearic acid in amounts from about 55% to about 80% by weight of the vitamin, in embodiments from about 60% to about 75% by weight of the vitamin, and magnesium oxide may be encapsulated with mono-glycerides and/or diglycerides, in amounts from about 50% to about 70% by weight of the magnesium oxide, in embodiments from about 55% to about 65% by weight of the magnesium oxide.

The melt temperatures of these encapsulants may be from about 120° F. to about 150° F.

The rapidly dissolving orally administrable palatable powder may also include natural flavors. Natural flavors that may be used in accordance with the present disclosure include any flavors capable of masking bitter tastes or enhancing sweetness. Natural flavors include, for example: masking flavors, masking powder, bitter masker; sugar flavor, natural flavor cane sugar type, vanilla and cream flavors, chocolate, fruit flavors such as SD natural Saskatoon berry flavor, natural mixed berry flavor, Marion Blackberry WONF, Blueberry WONF, Dark Sweet Cherry WONF, RSP Cherry WONF, Cranberry WONF, Cranberry WONF, Black Currant WONF, Concord Grape WONF, Clear Passionfruit WONF, Cloudy Passionfruit WONF, Peach WONF, Plum WONF, combinations thereof, and the like. Where some flavors, for example vanilla, cream, and/or chocolate flavors are added, an acid such as an organic acid described herein may not be required.

In embodiments, the powder may also include artificial flavors. Any artificial flavor within the purview of those skilled in the art, which does not negatively impact flavor, mouthfeel, or dissolution rate of the powder of the present disclosure, may be utilized.

In embodiments, the flavors for use with a powder of the present disclosure may be selected based upon the target audience and/or consumer of the powder. For example, for a powder to be administered to children, cotton candy or bubble gum flavors may be utilized. For prenatal usage, a lemon or other fruit flavor may be used. For adults, a mixed berry flavor may be used, etc. Regardless of the actual flavor, the end goal is to naturally mask the notes, aftertaste, bitterness of the vitamins, minerals, herbs, etc. used in any formula and provide the consumer a pleasant tasting formula suited to their general wants.

In embodiments, the amount of these flavors present in the rapidly dissolving orally administrable palatable powder may be from about 3% to about 15% by weight of the powder, in embodiments from about 5% to about 13% by weight of the powder.

In embodiments, as the dosage form is a powder, it may be desirable to include an anti-dust ingredient to minimize loss of product and/or ingredients during formation of the powder dosage form of the present disclosure. Suitable anti-dust ingredients are within the purview of those skilled in the art and include, for example, maltodextrin, or anti-caking agents such as silicas, combinations thereof, and the like. In embodiments a suitable anti-dust ingredient includes a maltodextrin derived from tapioca, such as N ZORBIT M, commercially available from National Starch. Such an anti-dust agent may be present in amounts from about 0.1% to about 5% by weight of the powder dosage form, in embodiments from about 0.5% to about 3.5% by weight of the powder dosage form, in embodiments about 2% by weight of the powder dosage form. Where an anti-caking agent such as silica is used, it may be present, in embodiments, in an amount up to about 2% by weight of the powder dosage form, in embodiments from about 0.1% to about 2% by weight of the powder dosage form, in embodiments from about 0.5% to about 1.5% by weight of the powder dosage form.

The rapidly dissolving orally administrable palatable powder may be formed by formulation and/or processing methods within the purview of those skilled in the art. Formulation and processing methods include, for example, agglomeration, wet granulation, spray granulation, microencapsulation, spray drying, fluid bed granulation, melt granulation, blending, dry blending, milling, crystallization, combinations thereof, and the like.

In embodiments, the rapidly dissolving orally administrable palatable powder may be formed by a dry blending process. Dry blending may occur by combining the ingredients in a suitable mixing or blending device within the purview of those skilled in the art, at mixing speeds of from about 8 revolutions per minute (rpm) to about 30 rpm, in embodiments from about 10 rpm to about 25 rpm, for a period of from about 2 minutes to about 20 minutes, in embodiments from about 5 minutes to about 15 minutes. Suitable devices for blending may include, in embodiments, those commercially available and sold as TOTE® blenders (from Tote Systems International), TURBULA® blenders (from Willy A. Bachofen), bin blenders (from Patterson Kelly Blenders), and the like.

The particle size distribution of the powder of the present disclosure may vary. For example, in embodiments, where the powder of the present disclosure includes both mannitol and xylitol, about 85% of the particles will have a particle size of less than about 315 microns.

The rapidly dissolving orally administrable palatable powder may then be divided into individual dosages. A dosage may be from about 0.5 grams to about 6 grams of powder, in embodiments from about 1 gram to about 5 grams of powder.

Administration of the rapidly dissolving orally administrable palatable powder may be in any manner. An individual dosage may be placed in the mouth, in embodiments, poured onto the tongue. The saliva in the mouth and/or on the tongue may dissolve the rapidly dissolving orally administrable palatable powder. In embodiments, the rapidly dissolving orally administrable palatable powder dissolves in the mouth within a period of from about 2 to about 30 seconds after placement in the mouth, in embodiments from about 5 to about 20 seconds after placement in the mouth, dependent on the amount of powder poured on the tongue.

The following Examples are being submitted to illustrate embodiments of the present disclosure. These Examples are intended to be illustrative only and are not intended to limit the scope of the present disclosure. Also, parts and percentages are by weight unless otherwise indicated.

### EXAMPLES

### Example 1

A powder dosage was prepared with the ingredients listed below in Table 1.

TABLE 1

| | Grams/ | % By Weight of |
|---|---|---|
| Ingredient | packet | Total Dosage |
| Mannitol, PEARLITOL ™ 300DC | 0.969 | 24.225 |
| (commercially available from Roquette, | | |
| Geneva, IL) | | |
| Vitamin and Mineral Premix FT 101168 | 0.895 | 22.375 |
| (see Table 2 below for details) | | |
| Xylitol, XYLISORB ™ 300 | 0.820 | 20.500 |
| (commercially available from Roquette, | | |
| Geneva, IL) | | |
| DESTAB Calcium Carbonate 95MD | 0.587 | 14.675 |
| Ultra (commercially available from | | |
| Particle Dynamics Inc. St. Louis, MO) | | |
| DESCOTE ™ Magnesium Oxide 40% | 0.219 | 5.475 |
| (commercially available from Particle | | |
| Dynamics Inc. St. Louis, MO) | | |
| Crospovidone NF (polyvinyl | 0.163 | 4.075 |
| pyrrolidone) | | |
| Masking Powder NFF #29879K | 0.008 | 0.200 |
| Bitter Masker SC015478 | 0.015 | 0.375 |
| Natural Flavor Cane Sugar Type, 8- | 0.200 | 5.000 |
| CSR-0680 | | |
| Malic Acid FCC Fine | 0.046 | 1.150 |
| SD Natural Saskatoon Berry Flavor | 0.008 | 0.200 |
| Type #25975 | | |
| Nat. Mixed Berry Fl WONF NV-32,794 | 0.015 | 0.375 |
| Silicon Dioxide SYLOID ® 244 from | 0.020 | 0.498 |
| Grace) | | |
| Thaumatin, TALIN ™ MD90 | 0.035 | 0.875 |
| (commercially available from Overseal, | | |
| NY, NY) | | |

The Vitamin and Mineral Premix utilized in the powder is set forth below in Table 2.

TABLE 2

| Vitamin and Mineral | | | |
|---|---|---|---|
| Premix (referred to | Feasible | Amount Per 4 g | % |
| herein as FT101168) | Amounts | Packet (units) | RDI |
| Vitamin A | Up to 9990 IU | 5000 IU | 100 |
| Vitamin C | Up to 2000 mg | 100 mg | 167 |
| Vitamin D | Up to 2000 IU | 1000 IU | 250 |
| Vitamin E | Up to 1490 IU | 100 IU | 333 |
| Thiamin | Up to 200 mg | 1.5 mg | 100 |
| Riboflavin | Up to 400 mg | 25 mg | 1471 |
| Niacin | Up to 35 mg | 20 mg | 100 |
| Vitamin B6 | Up to 100 mg | 25 mg | 1250 |
| Folate | Up to 1000 mcg | 400 mcg | 100 |
| Vitamin B12 | Up to 1000 mcg | 50 mcg | 833 |
| Pantothenic Acid | Up to 1200 mg | 60 mg | 600 |
| Biotin | Up to 200 mg | 300 mcg | 100 |
| Calcium | Up to 1500 mg | 200 mg | 20 |
| Chromium | Up to 1000 | 120 mcg | 100 |
| Copper | Up to 10 mg | 0.5 mg | 25 |
| Iodine | Up to 1100 mcg | 150 mcg | 100 |
| Iron | Up to 45 mg | 1.8 mg | 10 |
| Magnesium | Up to 350 mg | 50 mg | 13 |
| Manganese | Up to 11 mg | 2.3 mg | 115 |
| Molybdenum | Up to 2000 mcg | 75 mcg | 100 |
| Selenium | Up to 400 mcg | 56 mcg | 80 |
| Zinc | Up to 40 mg | 20 mg | 133 |
| Boron | Up to 20 mg | 0.1 mg | n/a |

IU = international unit
mg = milligram
mcg = micrograms

The ingredients above were mixed in a dry blending process utilizing a bin blender (from Patterson Kelly Blenders), and the mixing parameters (speed and time) described above, i.e., by combining the ingredients at mixing speeds of from about 10 rpm to about 25 rpm, for a period of from about 5 minutes to about 15 minutes. The blended powder was then packeted in 4 gram packets. The amounts of the various ingredients in the powder in the 4 gram packets are set forth above in Tables 1 and 2.

### Example 2

Another powder was prepared following the general procedure described above in Example 1, with the ingredients listed below in Table 3.

TABLE 3

| | | |
|---|---|---|
| Copper Amino Acid Chelate (Cu 10%) | 5.789 | mg |
| Vitamin A Palmitate, 250,000 Iu/G (USP-FCC) | 25 | mg |
| Biotin 1% Trit (USP) | 40.5 | mg |
| Calcium Ascorbate (2 H2O) (USP-FCC) | 160.355 | mg |
| Folic Acid (90% Fa) (USP) | 0.6 | mg |
| Niacinamide 33 ⅓% | 76.687 | mg |
| Pyridoxine HCl 33 ⅓ (USP) | 114.093 | mg |
| Vitamin B1 Mono 33 ⅓% (USP) | 5.752 | mg |
| Vitamin B12 1.0% Trit (USP) | 6.75 | mg |
| Vitamin B2 33 ⅓% (USP) | 98.767 | mg |
| Vitamin D2 400 Iu/Mg (USP) | 3.125 | mg |
| Vitamin E 700 Iu/G (USP-FCC) | 178.571 | mg |
| Boron Amino Acid Complex (B 10%) | 1.1 | mg |
| Calcium Carbonate 95 Md Ultra 250 (Destab) | 587 | mg |
| Chromium Polynicotinate 10% Chromemate | 1.412 | mg |
| D-Calcium Pantothenate (USP-FCC) | 82.418 | mg |
| Ferric Amino Acid Chelate | 10.53 | mg |
| Magnesium Oxide 40% Ctd | 256.696 | mg |
| Manganese Amino Acid Chelate (Mn 16%) | 15.975 | mg |
| Potassium Iodide 10% (USP-FCC) | 2.256 | mg |
| Selenium Yeast 2000 Mcg/Gm | 32.2 | mg |
| Sodium Molybdate (2 H2O) | 0.218 | mg |
| Zinc Oxide (USP) | 28.513 | mg |
| Crospovidone | 163 | mg |
| Malic Acid (FCC) | 46 | mg |
| Mannitol (USP) | 933.193 | mg |
| Masking Flavor, Bitter | 15 | mg |
| Natural Cane Sugar Flavor | 200 | mg |
| Natural Masking Powder | 8 | mg |
| Natural Mixed Berry Flavor | 16.5 | mg |
| Natural Saskatoon Berry Flavor | 9 | mg |
| Silicon Dioxide | 20 | mg |
| Talin | 35 | mg |
| Xylitol | 820 | mg |

### Example 3

Another powder was prepared following the general procedure described above in Example 1, with the ingredients listed below in Table 4.

TABLE 4

| | |
|---|---|
| Vitamin A Palmitate 250,000 IU/g (USP-FCC) | about 13 mg |
| Vitamin D2 400 IU/mg (USP) | about 2 mg |
| Vitamin E 700 IU/g USP-FCC | about 54 mg |
| Biotin 1% Trit | about 7 mg |
| Folic Acid (90% Folic Acid) (USP) | about 1 mg |
| Niacinamide 33-⅓% | about 77 mg |
| D-Calcium Pantothenate (USP-FCC) | about 14 mg |
| Vitamin B1 Mononitrate 33 ⅓% (USP) | about 6 mg |
| Vitamin B2 33 ⅓% (USP) | about 7 mg |
| Pyridoxine HCl 33-⅓% (USP) | about 9 mg |
| Vitamin B12 1.0% Trit (USP) | about 1 mg |
| Calcium Ascorbate (2 H2O) (USP-FCC) | about 95 mg |
| Potassium Iodide 10% (USP-FCC) | about 2 mg |
| Manganese Amino Acid Chelate (Mn 16%) | about 7 mg |
| Zinc Oxide (USP) | about 21 mg |
| DESTAB Calcium Carbonate 95MD Ultra | about 405 mg |
| DESCOTE Magnesium Oxide 40% | about 219 mg |
| Mannitol, Pearlitol 160C | about 644 mg |
| Xylitol | about 610 mg |
| Fibersol 2 | about 82 mg |
| Masking Powder | about 4 mg |
| Malic Acid, Fine | about 25 mg |
| Nat Bubble Gum Flavor | about 18 mg |
| Nat. Strawberry Flavor | about 50 mg |
| Bitter Masker | about 8 mg |
| Nat. Flavor Cane Sugar Type | about 100 mg |
| Talin | about 11 mg |
| Silicon Dioxide | about 13 mg |

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A composition comprising a powder including:
at least one bulking agent;
at least one sweetening ingredient; and
at least one active ingredient selected from the group consisting of Vitamin A, Vitamin C, Vitamin D, Vitamin E, Thiamin, Riboflavin, Niacin, Vitamin B6, Folate, Vitamin B12, Pantothenic Acid, Biotin, Chromium, Copper, Iodine, Iron, Manganese, Molybdenum, Selenium, Zinc, Boron, omega-3 fatty acids, coenzyme Q10, calcium, probiotics, and combinations thereof,
wherein the powder dissolves in the mouth upon contact with saliva.

2. The composition of claim 1, the powder further comprising a flow agent selected from the group consisting of sodium bicarbonate, sodium ferrocyanide, postassium ferrocyanide, calcium ferrocyanide, bone phosphate, sodium silicate, silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane, and combinations thereof.

3. The composition of claim 1, wherein the at least one bulking agent is selected from the group consisting of polyols, sucrose, maltodextrin, and combinations thereof.

4. The composition of claim 3, wherein the polyol is selected from the group consisting of mannitol, xylitol, sorbitol, maltitol, erythritol, isomalt, lactitol, polyglycitol, and combinations thereof.

5. The composition of claim 1, wherein the sweetening ingredient is selected from the group consisting of stevia, sucralose, aspartame, saccharin, acesulfame K, sucrose, fructose, maltose, dextrose, thaumatin, and combinations thereof.

6. The composition of claim 1, wherein the bulking agent is present in an amount from about 40 percent by weight to about 80 percent by weight of the powder.

7. The composition of claim 1, further comprising an organic acid selected from the group consisting of malic acid, citric acid, fumaric acid, adipic acid, tartaric acid, acetic acid, lactic acid, phosphoric acid, sodium acid sulfate, and combinations thereof.

8. The composition of claim 1, wherein the at least one active ingredient comprises calcium in an amount from about 8% to about 23% by weight of the powder.

9. The composition of claim 1, wherein the active ingredient further comprises magnesium oxide.

10. The composition of claim 1, wherein the powder dissolves within a period of time from about 2 seconds to about 30 seconds after placement in the mouth.

11. A composition comprising a powder including:
at least one bulking agent in an amount of from about 40% to about 80% by weight of the powder;
at least one active ingredient in an amount of from about 10% to about 50% by weight of the powder;
calcium in an amount from about 12% to about 18% by weight of the powder;
at least one flow agent in an amount of from about 0% to about 2% by weight of the powder;
at least one sweetening ingredient in an amount of from about 0.5% to about 25% by weight of the powder;
at least one organic acid in an amount of from about 0.5% to about 5% by weight of the powder; and
at least one soluble fiber in an amount of from about 1% to about 25% by weight of the powder,
wherein the powder dissolves in the mouth upon contact with saliva.

12. The composition of claim 11, wherein the powder dissolves within a period of time from about 2 seconds to about 30 seconds after placement in the mouth.

13. A method comprising:
preparing a dietary supplement by dry blending a mixture comprising at least one bulking agent, at least one sweetening ingredient, and an active ingredient selected from the group consisting of Vitamin A, Vitamin C, Vitamin D, Vitamin E, Thiamin, Riboflavin, Niacin, Vitamin B6, Folate, Vitamin B12, Pantothenic Acid, Biotin, Chromium, Copper, Iodine, Iron, Manganese, Molybdenum, Selenium, Zinc, Boron, omega-3 fatty acids, coenzyme Q10, calcium, probiotics, and combinations thereof, to form a rapidly dissolving orally administrable palatable powder;
introducing the orally administrable powder into a mouth of a consumer; and
allowing the orally administrable powder to dissolve in the mouth.

14. The method of claim 13, wherein the orally administrable palatable powder dissolves in saliva.

15. The method of claim 13, further comprising packeting the rapidly dissolving orally administrable powder.

16. The method of claim 13, wherein the orally administrable powder dissolves over a period of time from about 2 seconds to about 30 seconds after placement in the mouth.
